(19) United States
(12) Patent Application Publication
(10) Pub. No.: US 2006/0169586 A1
(43) Pub. Date: Aug. 3, 2006

(54) Production line and treatment for organic product

(75) Inventors: Li Zhang, Belmont, MA (US); Yongchang Zheng, Watertown, MA (US); Russell J. MacDonald, Wilmington, MA (US); Yuander A. Ju, Andover, MA (US)

Correspondence Address: Schwegman, Lundberg, Woessner & Kluth, P.A., P.O. Box 2938, Minneapolis, MN, 55402, US

(73) Assignee: GE Ionics, Inc.

(21) Appl. No.: 11/292,796

(22) Filed: Dec. 2, 2005

Related U.S. Application Data
(63) Continuation of application No. PCT/US05/09312, filed on Mar. 17, 2005.
(60) Provisional application No. 60/553,753, filed on Mar. 17, 2004.

(51) Int. Cl. C07K 1/26

(52) U.S. Cl. 204/450

(57) ABSTRACT

A fluid from a fermentation process or the like is passed or circulated through chambers of a bipolar membrane electrodialysis unit to separate an ionizable organic acid stream and at least one co-ion or residual stream. The organic acid stream is preferably concentrated (e.g., by recirculation, dewatering or both), and a product is recovered from the concentrated stream, for example by crystallization, and other outputs from the electrodialysis unit may be integrated with overall treatment and applied elsewhere in the treatment system. Depleted feed may be returned upstream to enhance yield, condition the medium or form a by-product. Treatment systems of the invention may replace a cation exchange bed and/or various filter arrangements, and recirculation of the feed and product flows through the unit enhance recovery, separation and quality of the target species. An ED chamber may include a filling of ion exchange beads to maintain a desired operating efficiency as the feed is depleted, and the straight-through operation effectively operates as pre-filtration stage to provide downstream product-bearing flows with processing characteristics for enhanced treatment, recovery and product quality. When operated to treat a downstream waste, systems allow additional recovery of value in the form of product, unexpended nutrients, co-factors and/or other components present in the waste.

This application is a continuation under 35 U.S.C. 111(a) of PCT/US2005/009312, filed on Mar. 17, 2005, and published in English on Sep. 29, 2005, as WO 2005/089513, which claims the benefit under 35 U.S.C. 119(e) of U.S. Provisional Application Ser. No. 60/553,753, filed Mar. 17, 2004, which applications and publication are incorporated herein by reference.

### TECHNICAL FIELD

The present invention relates to industrial processes for production of bulk chemicals, and to the treatment or processing of an aqueous stream containing organic material, such as a product stream comprising as a relevant component thereof, one or more complex salts or ionizable components, such as a salt of an organic acid. In particular it relates to treatment systems employing an electrodialysis (ED) treatment unit and/or a bipolar membrane-type electrodialysis (BPED) treatment unit, and to products produced thereby. It relates quite generally to processes for separation, treatment or refining of fermentation product streams, plant or animal extraction streams, enzymatically-produced product- or intermediate-bearing streams, streams of chemically modified material derived from one of the foregoing sources, or other bulk streams containing ionizable organic components. For simplicity of exposition, these shall be referred to herein as “fermentation product stream”. These streams will, as a rule, include a target organic material as a significant component, typically appearing in a mixture with other components that may also be addressed by the treatment process.

### BACKGROUND OF THE INVENTION

Many simple chemicals are produced on an industrial scale by processes of fermentation, microbial or chemical digestion or other mechanism, from material such as plant syrup or milling byproducts, milk, corn, soy or other agricultural matter that is available in great quantity, sometimes as the waste material from another harvesting or extraction process. Common examples of such chemicals include various carboxylic acids, such as tartaric, acetic, maleic, ascorbic acid, and other simple organic materials, as well as specialty chemicals or chimeric homovariants (like L-lactic acid), that may be present in or efficiently produced from the bulk matter using enzymes or special strains of industrially useful organisms. An end chemical may be produced directly in a fermentation process, or may result from reaction or processing of a ketone or other precursor that is produced from products of such fermentation. Typically, one or more stages of post-fermentation processing are required to extract, modify, concentrate or refine the desired product or intermediary from the fermentation stream. Such processing may include a filtration process such as ultrafiltration to remove high molecular weight (e.g., protein) and other potentially interfering material, an ion exchange process to remove divalent metals, decolorize, acidify or otherwise condition the stream; acid, base or chemical addition to condition the feed or to effect chemical modification, and other processes to change pH, remove or substitute minerals. A process may also include steps such as nanofiltration to concentrate the stream and/or separate unwanted species or components; processes to cleave or add portions of the molecular structure, and processes to precipitate or crystallize the product, and to clarify or otherwise modify the stream.

Typically, the relevant organic compound, for example, a form of a lactic, ascorbic or simple aliphatic acid, is present together with a certain residual amount of the starting material and nutrients, as well as metabolic products of the fermentation process, so that various sugars, alcohols, ketones or acids, and other compounds may be present in the stream. A target component or desired product is frequently present as, or is predominantly converted to, an ionizable salt at one stage of the processing. Recovery of product from the salt may be effected by separating ionizable components from solution using electrodialysis, i.e., electrically separating and driving relevant materials through ion-selective membranes into an output channel.

Some early systems of this type, as shown in U.S. Pat. No. 2,921,005 (1960) and No. 4,057,483 (1977) employed basic chamber constructions made of multiple cation exchange membranes, rather than the more common alteration of cation and anion exchange membranes generally used in electrodialysis “stacks”, and sometimes utilized multiple three- or four-chamber basic units to form stacks that provided suitable sources for protonation of the organic moiety or hydroxylation of the inorganic ion, while efficiently separating the soluble ionic parts of the salt.

The use of ion-selective membranes in these prior art constructions effected conversion of an organic acid salt to an acid and a base by providing separate cells or flow chambers in which protonation of the acid moiety could be effected. However, differences in transport number of the cationic and anionic components would generally impede complete separation with standard electrodialysis cell construction, and many arrangements were proposed with three- or four-chamber constructions, in which circulation (to increase concentration in or transfer of ions from), or dilute streams (to decrease back˜diffusion) could be run in various chambers to enhance overall effectiveness. With the development of commercial bipolar (“water splitting”) membranes, such electrodialysis units and treatment regimens could be modified to incorporate at least one bipolar (BP) membrane in their basic cell structure. This construction was intended to generate localized excesses of the hydronium and hydroxide ions needed for the respective anion- and cation-receiving sub-chambers, and to more effectively block entry of unwanted species. Effective architectures using BP membranes were able to obtain respectable yields in simple two- or three-chamber constructions, efficiently splitting water in the BP membrane at a chamber boundary. Concentration of the acid or base recovered by such bipolar electrodialysis units could be achieved by suitable control of the flow rates and recirculation of the streams in the chambers.

By way of example, recovery of organic acids from corresponding salts or mixtures of material are described in the 1988 U.S. Pat. No. 4,781,809 of J. Falcone, Jr. Several separation/conversion processes and some ED unit designs are described in that patent, as well as in the 1989 bipolar membrane patent, No. 4,851,100 of inventors Hodgdon and Alexander. A useful overview of water splitting membrane electrodialysis technology around that period is found in the article Electrodialysis water splitting technology by K. N. Mani, in J. Membrane Sci., 58 (1991) 117-138. In that article, the author discussed useful process and efficiency considerations, sketched a number of simple multi-chamber basic cells useful in bipolar electrodialysis stack construction utilizing different arrangements of ion exchange membranes, and also indicated a number of features and advantages relevant to integration of bipolar membrane-based electrodialysis treatment processes into a conventional product processing or treatment line, such as those previously employed in treating waste streams or processing fermentation products.

A number of factors in the 1990 time period when the Mani article appeared—such as a desire to reduce chemical consumption or diminish chemical waste streams (as compared to processing steps involving strong acid treatment and/or exchange beds with their concomitant chemical regeneration requirements)—appeared to weigh in favor of incorporating such BPED treatment units into a number of existing production line or treatment applications. In the intervening decade, however, relatively few large scale processing plants have been constructed with bipolar electrodialysis treatment units.

A number of factors appear to be responsible for the slow adoption of BPED treatment technology. Commercially available lines of bipolar membranes have remained rather expensive, and while electrical splitting efficiency and current capacity of these membranes appear good, economic considerations have limited the industrial acceptance of BPED processing systems to a few higher-value applications or to small experimental and/or environmental niches. Competing processes, such as filtration, ion exchange and precipitation are mature and proven technologies, and the bulk cost of acid and caustic for chemical treatment or ion exchange regeneration have remained low.

This has probably also slowed the adoption of bipolar electrodialysis technology by most bulk chemical commodity and separation industries to which BPED processes would otherwise appear technically well suited. The general nature of bulk fermentation and similar chemical production processes, which commonly involve many plant-specific details and carry the likely presence of potentially fouling or interfering biological components, has undoubtedly also been an obstacle, because these factors suggest that substantial investment of research, piloting and trouble-shooting might be required to bring any specific application into fully controlled production. Perhaps also, because many mills or chemical producers effectively constitute large private empires that maintain close control over all information relevant to their products and production processes, detailed process information, and the necessary experience and expertise have not been widely shared with or made available to equipment and membrane suppliers. Thus, many factors may be cited for the apparently limited adoption of bipolar treatment technology.

In this state of affairs, there remains a need to improve processes for producing and treating bulk or specialty chemicals.

In particular, there remains a need for processes wherein BPED is integrated in a process line to reduce chemical or energy consumption, lower capital requirements, enhance yield or quality of a product or by-product, or otherwise improve the overall production or treatment process.

### SUMMARY OF THE INVENTION

One or more of these and other desirable ends are achieved according to the present invention, in a process and system wherein organic matter, such as that derived from a fermentation process, is treated as a batch or stream containing one or more organic components in a fluid medium. The medium, preferably filtered, e.g., by ultrafiltration or the like, is passed or circulated with the organic matter in salt form through a bipolar membrane electrodialysis unit to separate an ionizable organic acid stream and a co-ion stream. The organic acid stream is preferably concentrated (e.g., by recirculation, by dewatering or both), and the desired acid product is recovered from the concentrated stream, by a process such as crystallization. Advantageously, the ED treatment may produce several streams, and these may be integrated with the overall treatment system. Furthernore, the overall treatment may involve one or more chemical modification steps, with concentrated product flows of different organic salts at the different stages, any of which may be treated by electrodialysis. In one embodiment of a treatment line of the present invention, a bipolar electrodialysis assembly replaces the cation exchange media bed of a conventional process line design, and operates to produce an organic acid stream and an inorganic or weak organic base stream. The base stream (for example, caustic or ammonium hydroxide) is preferably applied elsewhere in the treatment system, for example to condition the medium or modify a component in a fermentation or product modification stage. The feed may be recirculated to extract a high yield of the target species, and the feed- or product-receiving chamber may include a filling of ion exchange beads to maintain a high operating current through the stack even as resistivity otherwise rises with the progressive depletion of the circulating fluid over time.

In another or further process, the bipolar membrane electrodialysis unit is assembled with plural three-chamber repeating units, and is arranged to receive the feed stock in its second chambers. The second chambers may include ion exchange beads as described above, which may be of mixed or other type, as appropriate to the projected conditions. In operation, the unit transfers to and concentrates a desired component in the first chambers, providing an acid-enriched output stream, while passing undesired and non-ionized components straight through the second chambers as a depleted stream (e.g., depleted of the target product). The depleted stream may, for example, contain large molecules, alcohols, sugars and other non-ionized or poorly ionized material. Metal ions are transferred into the third chambers, the output of which (such as recovered caustic or trace nutrient species) may in certain cases be applied to other stages of the process line to enhance efficiency of the overall treatment and to effect certain cost savings.

Product may be recovered from the acid-enriched output stream of the first chambers, for example, by evaporation, crystallization or the like. Advantageously, the three-chamber bipolar ED in this embodiment, in addition to isolating and concentrating the target product in acid form, separates the product-carrying flow from many residual and impurity components retained in the depleted feed stream, and thus simultaneously operates as pre-filtration stage that advantageously provides different characteristics than those of a conventional filter-based or exchange-bed based treatment system in which physical pore size or binding affinities govern treatment. This is highly useful, because by diverting the large and the non-ionic components from the flow that passes to subsequent product treatment steps, the target material passed to downstream product treatment processes is a purer, or less contaminated product-bearing stream, and the downstream units therefore may achieve higher recovery, or a purer recovery, or produce smaller waste streams. Thus, for example, residual waste from a downstream product crystallization or other recovery step is advantageously reduced, and, in addition, all or a portion of the straight-through-depleted feed stream may be fed back to the underlying fermentation or other upstream process to maximize digestion of the included nutrients or other treatment of the raw stream, thereby increasing product yield. When depleted feed is returned to the fermentation or earlier stage, the returned portion may also be partially distilled or otherwise treated, if necessary, or a bleed may be set at an effective rate, to reduce the concentration of or to remove accumulated components such as metabolites or toxins in the feedback stream or fermentation vat below a level that might otherwise adversely affect the fermentation.

In yet another or further embodiment, an ED or BPED stage, or both, are placed to treat a waste stream remaining after a recovery step, such as the precipitation or crystallization of a product or intermediate, and the electrodialysis treatment operates to transfer remaining ionizable acid components into a recovery stream while passing non-ionized or opposite-charge components into one or more other streams such as a waste stream of lesser volume. In accordance with this aspect of the invention, the ED and/or BPED recovery process is applied at a downstream process end, and the recovery stream, which may be or may include recovered organic acid, base, or nutrient and trace mineral components, may be returned to an upstream process stage to increase yield.

### BRIEF DESCRIPTION OF THE DRAWINGS

These and other features of the invention will be understood from the description and claims herein, taken together with the drawings illustrating details and representative embodiments of the invention, wherein:

FIG. IA illustrates a prior art treatment process for production of bulk organic acid by refinement of fermentation product liquor;

FIG. IB schematically depicts a treatment or production process and processing line in accordance with the present invention;

FIG. 2 schematically depicts operation on a feed stream in a three-chamber bipolar ED unit in accordance with the present invention; and

FIGS. 3A and 3B show different two-chamber units and corresponding modes of treatment for systems of the invention.

### DETAILED DESCRIPTION

The invention is best understood following a brief discussion of a prior art system, which serves to illustrate some characteristics of an organic process stream and relevant treatment modalities and process considerations. FIG. 1A shows a system 10 of the prior art which operates on a product stream 1a produced by an upstream or initial biological production process, e.g., a biosynthesis, culture, enzymatic modification or fermentation process 2, shown schematically, with downstream processes 4 that operate to modify, separate and/or concentrate and purify a product therefrom. The initial or upstream process 2 may be rather simple or quite complex, depending upon biological process considerations, the starting sugars or other materials and the strains of fermentation organisms involved, and any bulk treatments or chemical modifications that may be required to adapt the feed material to the culture, or the fermentation product stream to a desired intermediate. Generally, process 2 requires a controlled series of steps in one or more culture, conditioning and other vessels (not shown). Persons skilled in the art will appreciate the scope of such processes and details involved in each. For purposes of the present disclosure it suffices to generically denote the upstream fermentation process 2.

The processes and operations designated 2 may include various direct chemical treatments or additions, for example to convert or transform the biomaterial by simple reaction such as esterification, conversion to a related salt or the like. The desired organic material from fermentation 2 appears in a stream 1a of process/product liquor, which, variously, may be withdrawn continuously or as a batch from the fermentation stage, and is treated by the processes 4. The stream 1a has one or more identified fermentation product components, in a suitable concentration such that the further treatments refine, produce or extract a more concentrated and relatively pure product from the stream.

The processes 4 may typically include filtration and/or ion exchange processes, a chemical modification process or a product separation process. By way of example, one process for the production of vitamin C is to transform and ferment simple sugars or alcohols to provide a gulonic acid or salt intermediate, such as 2-keto-L-gulonate which is acidified and subject to esterification to form ascorbate. The ascorbate may be the desired end product, or it may be further converted to an acid form, as required. Many other bulk and specialty chemicals are produced by treatment steps via a gluconate, lactate or other intermediate or product thereof that has been derived, in part, by fermentation.

As further shown in FIG. 1A, one representative prior art downstream process 4 for treatment of the fermentation product liquor includes a first filtration stage, such as an ultrafiltration stage 12 that serves, inter alia, to retain (remove) certain material present in the liquor which could otherwise foul downstream treatment media. Ultrafiltration removes large molecule or proteinaceous material. This is followed by a cation exchange bed 14 that removes cations, lowering the pH, and a nanofiltration system 16 that allows water and dissolved (inorganic) salts to pass from the system, thereby retaining and concentrating the target acidified fermentation products in the retained liquor stream 1b. Thus concentrated, the stream may be subjected to processes such as chemical treatments or modification, or, if the target product is already present at this stage, the desired product may be directly recovered from the concentrated liquor 1b, e.g., by crystallization, evaporation or a combination of these steps. In the Figure, a product is crystallized in crystallizer 18, leaving a waste liquor 20, that may contains various minerals, sugars, alcohols or ketones and residual product that failed to crystallize in the preceding step.

FIG. 1A is thus intended to be broadly representative of a class of industrial processes for preparing a bulk chemical. In practical instances, different sub-stages may occur a number of times in the overall treatment sequence, e.g., to refine, condition or increase the concentration of a particular intermediate, to assure that the specific modification reactions primarily form a specific intended material, or achieve a desired environment or other characteristic or condition. Many different processes within this general framework may exist for producing a particular bulk chemical, depending on the starting materials involved. For example, the production of vitamin C may start with a number of different materials;

starting from glucose a suitable process may employ two fermentations to form sorbitol, then sorbose, a chemical conversion of the sorbose to 2˜keto-L-gulonate, conversion (e.g., by ion exchange) to gulonic acid, and esterification and further treatment in organic solvent to form the desired end product.

The present invention will now be illustrated in the context of treatment processes as described generally above.

FIG. IB illustrates by way of example a system 100 that implements a product recovery process in accordance with one aspect of the present invention, configured with one or more bipolar membrane electrodialysis treatment units. As shown in FIG. 1B, a system 10 includes a process line 4′ that operates on material from an upstream or initial biological production process, e.g., a biosynthesis, culture or fermentation process 2, shown schematically, to modify, recover and/or concentrate and purify a product therefrom. The fermentation process may be any known process that operates to produce an intended starting product, and typically involves a controlled growth arrangement wherein a bacterial or fungal culture produces material as a metabolic end-product under conditions of controlled growth in a nutrient medium. The relevant culture organisms may be retained in the fermentation vessel, and a supernatant or filtered flow containing the desired product removed, continuously or in a batch, to provide the product flow in a feed stream that is to be further treated. While the invention may be applied to implement extremely high value (pharmaceutical) product separations or treatments, it is advantageously applied using large-area electrodialysis apparatus to treat bulk chemical or specialty chemical production streams, and will be so described herein.

In accordance with one principal aspect of the invention, the medium 1a from process 2, preferably filtered or otherwise conditioned, e.g., by ultrafiltration, ion exchange or other steps, is passed or circulated with the organic matter in salt form through an electrodialysis system, that includes a bipolar membrane electrodialysis unit operated to separate an ionizable organic target into a stream of the target acid and a co-ion stream. The organic acid stream is preferably further concentrated (e.g., by recirculation, by subsequent dewatering or both), and a desired acid product is recovered from the concentrated stream, for example by crystallization, evaporation or other process, depending on the degree of purity desired and other factors. Economics of the concentration and recovery processes may have substantial impact on the overall treatment. Several aspects of the treatment according to the present invention provide benefits for this processing.

The ED unit may produce several streams, and these may be integrated with overall treatment. Thus inorganic ions removed from the ED feed may returned (as salts, acids or bases) to other steps, and non-ionic material in a depleted feed may be returned to an upstream utilization or downstream process.

In one embodiment of a treatment line of the present invention, a bipolar electrodialysis assembly, which may optionally be preceded by a conventional ED unit, replaces the cation exchange media bed of conventional process line designs (such as that of FIG. IA), and operates to produce an organic acid stream and an inorganic or weak organic base stream. The base stream (for example, caustic) is preferably applied elsewhere in the treatment system, for example to condition the medium or modify a component in one fermentation or product modification stage.

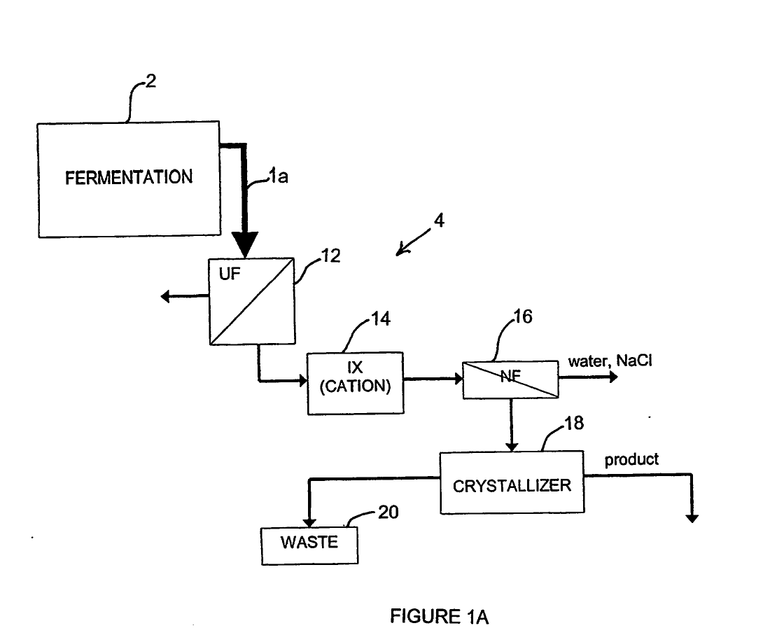

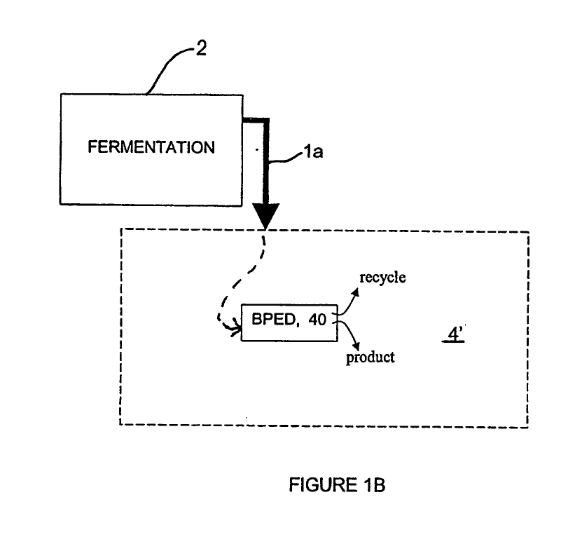

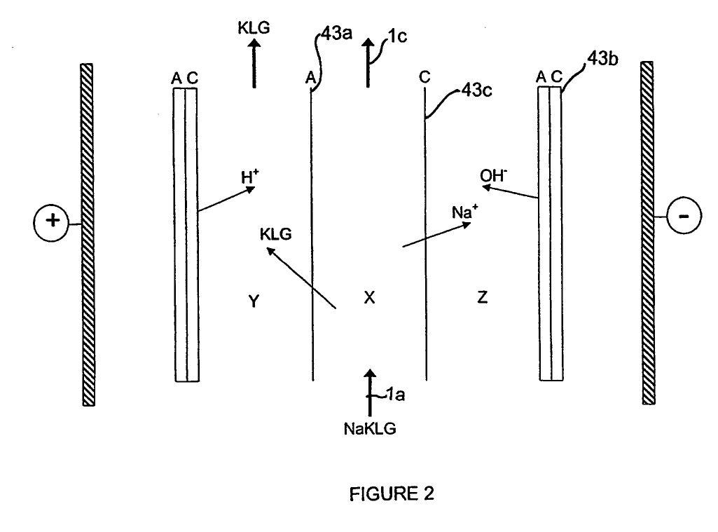

FIG. 2 illustrates one bipolar membrane electrodialysis (BPED) arrangement 40 for processing a material such as a keto-L-gulonate (KLG salt) in the stream 1a. As shown, the general architecture of the BPED stack 40 includes a cathode 41 at one end, an anode 42 at the other end, and a plurality of ion exchange membranes 43a, 43b, 43c arranged in a regular sequence therebetween to define treatment or ion-receiving flow chambers. The membranes are of three exchange types, namely cation exchange membranes C (43c), anion exchange membranes A (43a), and bipolar (BP) membranes 43b. The bipolar membranes are here also labeled AC or CA to indicate their polarity or orientation relative to the electrodes in this construction. The basic arrangement defined by the sequence BP-A-C-BP forms repeating units of three chambers Y, X and Z, which are arranged in a stack, wherein suitable manifolds are provided to define three separate flows through the corresponding chambers. One or more additional membranes, as well as a spacer or other structure may be associated with each electrode chamber at the ends, as known in the art, to prevent various scaling and other electrochemical effects that occur under the electrolyzing conditions and chemical environment in the electrode compartments.

One embodiment of a system employing such a three-chamber bipolar electrodialysis assembly provides the feed stream (e.g., stream 1a) to the central chamber X, extracting 2KLG into chamber Y and the other salt ions (e.g., Na+ or NH₄+) into chamber Z. The 2KLG is acidified by hydronium ions from water splitting in membrane 43b bounding chamber Y, while the metal ions combine with hydroxyl ions produced by the bipolar membrane bounding chamber Z. In this arrangement, the outflow 1_{Y} from chamber Y is the desired product stream, while the outflow 1c of chamber X, namely the product-depleted portion of the feed stream 1a will contain certain sugars and material that is not ionized by the ED process. Thus, the unit 40 advantageously “filters out” such material from the treatment portion, stream 1_{Y}, facilitating the downstream purification steps. For example, such impurities are not passed to crystallizer (18, FIG. IA) and need not be dealt with in the crystallizer waste (20, FIG. 1A). The product-depleted stream 1c may be passed multiple times through the chamber X to maximize the removal of the desired 2KLG product into stream 1_{Y}, e.g., it may be recirculated batchwise to a desired endpoint or by recirculation of a portion thereof in a feedback loop. The depleted batch or non-recirculated portion of stream 1c may then be returned to the upstream fermentation process to maximize utilization of the nutrients remaining therein.

In a further embodiment of this aspect of the invention, a conventional electrodialysis (ED or EDR) unit may be provided as a first stage ahead of the bipolar ED unit, to perform an initial treatment step. In this case, the first stage ED is preferably operated to remove the cationic and anionic portions of the targeted organic salt into the first stage concentrate stream, and the concentrate from the first stage serves as the input feed to the bipolar process described above.

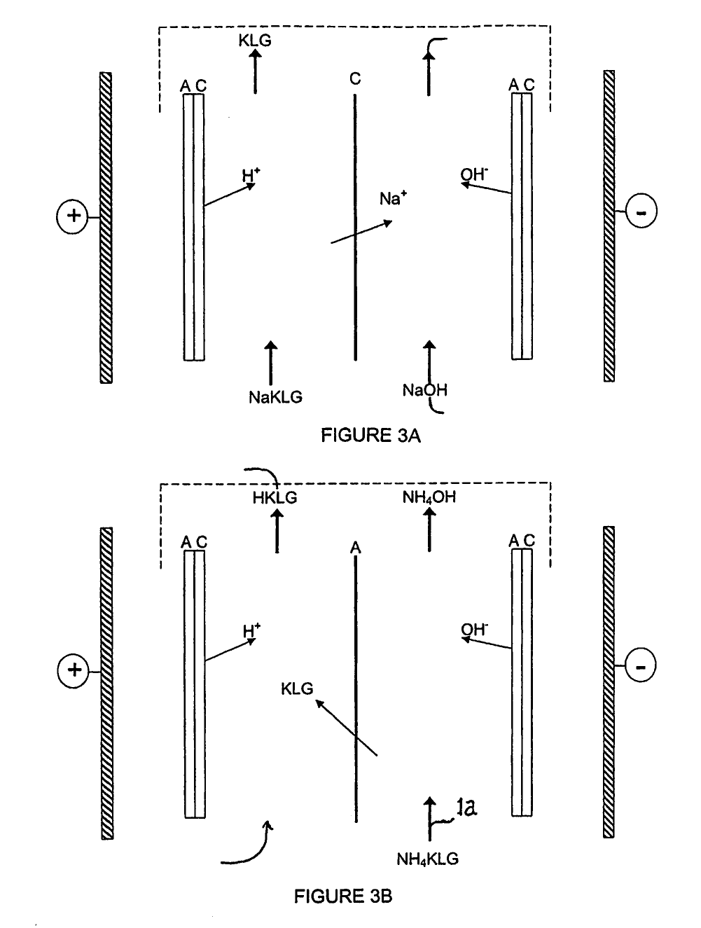

The BPED unit may also employ other cell constructions, with a single monotype exchange membrane (A or C) between two bipolar membranes to form a two-chamber bipolar cell architecture. Two such constructions are shown in FIGS. 3A and 3B, in which (continuing with a KLG salt example) either the KLG or the cation is transported out of the through-stream into the adjacent chamber.

Electrode cells at each end may have different or independent fluid circulation (not specifically illustrated). In any of these embodiments, one or both streams may be recirculated to reach a desired removal or concentration endpoint. Furthermore, a filling of ion exchange beads or fabric may be placed in one or more chambers to assure a sufficient conductivity to maintain the desired level of current in the stack as a whole. For removal of the target organic moiety, an anion exchange bead filling is preferred in the central chamber, whereas either anion or mixed-type may be employed in the product-receiving chamber. Use of exchange beads helps to maintain conductivity and efficient transport when the solution conductivity is low, and allows the feed to be recirculated through the central chamber to extract a maximum amount of the target species into the adjacent product acid-receiving chamber. Thus one or several chambers may contain exchange resin. Suitable resins may include macroporous resins and those having fouling resistance for comparable feed streams, specialty decolorizing resins, and others. Flows may also be treated or maintained at a suitable pH to minimize fouling, and to assure that the desired organic product is ionizable in the treatment cells.

In operation, when a three chamber unit receives the feed in its second chambers and transfers the desired component in the first chambers, to provide an acid-enriched output stream, the undesired and non-ionized components may pass straight through the second chambers as a depleted stream. The depleted stream may, for example; contain large molecules, alcohols, sugars and other non-ionized or poorly ionized material. Metal ions or other cations are transferred into the third chambers, the output of which (such as recovered caustic, weak base, certain nutrient or trace elements) may in certain cases be applied to other stages of the process line to enhance efficiency of the overall treatment and effect certain enhancements or efficiencies. By recirculation of the feed and the product streams at appropriate flow rates, concentration of the target product in the acid enriched output stream of the first chambers may be increased, and further concentration, for example, by evaporation, crystallization or the like, using processes similar to those of the prior art examples described above provides enhanced recovery or recovery of a more pure product. Advantageously, the bipolar ED in this embodiment, in addition to isolating and concentrating the target product in acid form, separates the product-carrying flow from most residual and impurity components which remain present in the depleted feed stream. In this sense, the BPED (as well as the first-stage ED treatment described above, when that is employed), operates as pre-filtration stage that advantageously provides different characteristics than a conventional filter-based or exchange-bed based pretreatment, in which physical pore size or charge characteristics largely determine the final stream composition. The present invention, by diverting the large and the non-ionic components from the flow that passes to subsequent product treatment steps, provides a purer, or less contaminated product-bearing stream to the downstream product treatment processes, promoting higher recovery, or a purer recovery, and/or generating a smaller amount of downstream waste.

Thus, for example, residual waste from a downstream crystallization or other recovery will advantageously be reduced, and the crystallizer liquor may be subjected to a second crystallization stage without extensive preconditioning. As noted above, all or a portion of straight-through depleted feed stream 1c may be fed back to the underlying fermentation or upstream process to maximize digestion of the included nutrients or other treatment of the raw stream. When depleted feed is returned to the fermentation or earlier stage, the returned portion may also be partially distilled or otherwise treated, if necessary, or a bleed may be set at an effective rate, to recover a by-product, or to limit the concentration of or remove an accumulated component, metabolite or toxin in the feedback stream or fermentation vat below a level that would adversely affect the fermentation.

This filtration/recovery aspect of the BP treatment systems of the invention may also be applied downstream of the principal treatment, either in a system as described above, or by performing such ED on a fluid at the post-crystallization or post-recovery stage of a conventional production plant. In accordance with this aspect of the invention, an ED or BPED stage, or both, are provided to treat the waste liquor remaining after a recovery step, such as precipitation or crystallization of a product or intermediate. For example such electrodialysis may be performed on the waste output 20 of the process in FIG. 1A.

As is known, such crystallizer waste liquor may contain significant amounts of unrecovered product (e.g., 2KLG) as well as sugars, alcohols, etc. A BPED treatment may transfer remaining ionizable acid components into a secondary recovery stream while passing non-ionized or opposite-charge components into one or more other streams such as a waste stream of lesser volume, or a cleaner residual nutrient stream for return to the process, or a secondary byproduct such as a feed additive or fertilizer. Treatment of the waste 20 may involve preconditioning, such as dilution, filtration and/or pH adjustment, and may be done in stages, e.g., with ED followed by BPED, if the nature of the waste 20 does not admit of a single stage or direct treatment. In accordance with this aspect of the invention, the waste, which may for example include substantial amounts of unrecovered product, as well as undigested nutrients, trace minerals and co-products, is treated by the ED/BPED units to recover additional ionizable product. Electrical operation on the relatively high concentration crystallizer waste stream can be quite efficient, and by cleaning up product or precursor from the crystallizer waste, the overall yield may be significantly enhanced, which can improve economics of the overall production process.

Among the other advantages achieved by the invention, it should also be observed that the production of a product stream and a re-usable co-stream allow great flexibility in addressing treatment economics. One or more savings in recovered nutrients, recovered acid, separation of a weak base or caustic stream, and reclaimed product waste may offset overall capital or maintenance expenses (e.g., for membranes, equipment and electricity), while the virtual filtration achieved by the various pass-through or interchamber transfer BPED configurations provides effective treatment and organic acid production with less capital investment, e.g., reducing the need for ultrafiltration or nanofiltration banks (12, 16 of FIG. 1A), or for exchange beds and regeneration chemicals. Processes of the present invention can produce a decolorized product of higher value, and thus may eliminate the need for an ion exchange or other clarifiers.

Several examples will serve to illustrate operating parameters and the general effectiveness of the described invention.

### EXAMPLE 1

### Purification & Recovery of Ascorbic Acid from Raw Sodium Ascorbate

A bipolar electrodialysis 9″×10″ stack was assembled having eight three-chamber units and two two-chamber units with an electrode chamber at each end of the stack. The effective area of each membrane was about 232 cm². The three-chamber unit included a bipolar membrane, a cation membrane (Ionics CR69EXMP) and an anion membrane arranged as described in FIG. 2. The two-chamber unit included one bipolar membrane and one cation membrane (Ionics CR69EXMP) arranged as described in FIG. 3A. The anion membrane used in the three-chamber units was an Ionics anti-fouling anion membrane (Ionics AR204SZRA) that allows organic ion to pass through. Ports were arranged so that in the three-chamber unit, feed solution of fermentation broth is passed through the middle chamber (X), the product of organic acid is passed through the left chamber (Y), and caustic solution passed through the right chamber (Z). In the two-chamber unit, the organic acid is passed through the left chamber while the caustic stream is passed through the right chamber. The three-chamber units act as purification and recovery of organic acid. The two-chamber units act to remove metal ions leak through bipolar membrane (co-ion leak) to lower the metal ion in the organic acid product.

Approximately 1000 g of dry raw sodium ascorbate from fermentation with purity of 88.1% were dissolved in 5 liters of pure water to get about 20% solution of raw sodium ascorbate. The solution was fed into the feed tank of the ED system and circulated in the chamber X at flow rate about 0.8 liter/min as shown in the FIG. 2. In the acid tank, 3 liter of water was added and circulated in the acid chamber. In the caustic tank, 3 liters of water was filled in and circulated into the caustic chamber and cathode chamber. In the anode chamber, 1% H₂SO₄ solution was circulated as the electrolyte. Conductivity of the feed solution was initially 24.7 mS/cm. The current density of the ED process was about 30 mA/cm² with overall voltage about 51-52 Volts across the stack, and treatment was carried out until conductivity of the feed solution dropped to about 0.5 mS/cm. The process took about 155 minutes.

The resulting ascorbic acid product solution was a very light yellow solution compared with the dark grey color of the feed solution. Yield was 88.0% based on ascorbate ion, and the current efficiency was 64%. When the product solution was concentrated and crystallized, product purity was 97.6% without sodium ion. It was believed that the 2.4% impurity might be largely oxidation products of ascorbic acid due to the drying process employed. Power consumption was about 1.1 kwh/kg ascorbic acid.

### EXAMPLE 2

### Purification and Recovery of Lactic Acid from Sodium Lactate

A bipolar electrodialysis 9″×10″ stack was assembled comprising five three-chamber units with an electrode chamber at each end of the stack. The effective area of each membrane was about 232 cm^{2,and} the three-chamber units had a bipolar membrane, a cation membrane (Ionics CR69EXMP) and an anion membrane (Ionics AR103QDP) arranged as described in FIG. 2. In the three-chamber unit, a feed solution simulating a fermentation broth was run through the middle chamber (X), the product of organic acid was run through the left chamber (Y), and caustic solution run through the right chamber (Z).

The feed solution used in this process example was a synthetic solution containing 9.2% of sodium lactate with sugar and protein similar to a fermentation broth. Three liters of feed solution were placed in the feed tank of the ED system and circulated in the chamber X at flow rate about 0.5 liter/min as shown in the FIG. 2. In the acid tank, 3 liters of water was added and circulated in the acid chamber. In the caustic/base tank, 3 liters of 0.2N sodium hydroxide solution was provided and circulated through the caustic chamber and the cathode chamber. In the anode chamber, 1% H₂SO₄ solution was circulated as the electrolyte solution. The conductivity of the feed solution was initially 34.2 mS/cm. The current density of the ED process was about 8-30 mA/cm² with overall voltage about 15-32 Volts across the stack with the process run until conductivity of the feed solution dropped to about 0.7 mS/cm. over the course of about 190 minutes.

Yield was about 94.3% with very little sugar and protein passing into the product, and the current efficiency was 88.8%. Power consumption was about 1.76 kwh/kg lactic acid.

The foregoing examples demonstrate efficient and effective organic acid separation, purification and conversion to acid form with desirable product characteristics.

The invention being thus disclosed and illustrative embodiments described, a number of variations and modifications thereof, as well as adaptations to other known treatment or production processes will occur to those of ordinary skill in the art. All such variations, modifications and adaptations are considered to be within the scope of the invention, and to be encompassed by the claims appended hereto.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A treatment system for operation in process line operating on an organic material feed stream or liquor containing a desired product or intermediate material together with large molecule/protein material, soluble mineral material and non-ionized material such as sugar, ketone or alcohol components, and wherein the process line includes a three-compartment electrodialysis unit wherein feed liquor is supplied to a second compartment, passing the desired product material into an adjacent third compartment and passing at least some soluble mineral material into a first compartment, a remainder of said feed passing through the second chamber and said unit being operative to retain the non-ionized and large molecule/protein material in said feed leaving the second compartment, said third compartment providing a concentrated and refined organic acid product output stream to a crystallizer, evaporator, recovery or conversion process and said second compartment providing a product depleted waste stream.

2. The treatment system of claim 1, wherein said product depleted waste stream includes toxic material that is thereby removed from the treatment production cycle.

3. The treatment system of claim 1, wherein said three compartment electrodialysis unit is positioned at an upstream treatment stage downstream of a fermentation process, and operates to provide said product depleted waste stream with included nutrients and intermediaries for return to an upstream or fermentation process.

4. The treatment system of claim 1, comprising an ED and/or a BPED electrodialysis unit positioned at a downstream treatment stage to treat waste liquor from a recovery or concentration process, and wherein the electrodialysis unit positioned at the downstream treatment stage operates to extract additional organic acid and provide an industrially-useful product-depleted waste-stream that may be returned to upstream fermentation or process stages.

5. The treatment system of claim 1, comprising ion exchange material in a said chamber to maintain conductivity.

6. The treatment system of claim 5, wherein the electrodialysis unit clarifies organic acid product produced by said process.

7. A treatment system for operation in process line operating on a feed stream or liquor containing a target organic desired product or intermediate material together with large molecule/protein material, soluble mineral material and non-ionized material such as sugar, ketone or alcohol components, and wherein the process line includes a three-compartment electrodialysis unit wherein feed liquor is supplied to a second compartment, passing the desired product material into an adjacent third compartment and passing at least some soluble mineral material into a first compartment, a remainder of said feed passing through the second chamber and said unit being operative to retain the non-ionized and large molecule/protein material in said feed leaving the second compartment, said third compartment providing a concentrated and refined organic acid product output stream to a crystallizer, evaporator, recovery or conversion process and said second compartment providing a product depleted waste stream, and wherein said feed stream is the concentrate stream of a first stage electrodialysis (ED) system that is substantially free of fouling organic material.
